(19) United States
(12) Patent Application Publication
(10) Pub. No.: US 2008/0064702 A1
(43) Pub. Date: Mar. 13, 2008

(54) Use of folates for the prevention and treatment of vascular diseases

(76) Inventors: Charalambos Antoniades, Athens (GR); Cheerag Shirodaria, Oxford (GB); Keith M. Channon, Oxford (GB); Rudolf Moser, Schaffhausen (CH)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/517,587

(22) Filed: Sep. 8, 2006

(51) Int. Cl. A61K 31/525

(52) U.S. Cl. 514/250; 514/251

(57) ABSTRACT

This invention relates to the use of folates for the prevention and/or treatment of cardiovascular diseases, such as atherosclerosis, and in particular for modulating endothelial nitric oxide synthase (eNOS). The invention further relates to pharmaceutical preparations consisting of said folates and a pharmaceutically acceptable carrier, optionally in combination with other pharmaceutically active agents, as well as therapeutic methods using said folates or pharmaceutical preparations thereof.

### FIELD OF THE INVENTION

This invention relates to the use of folates for the prevention and/or treatment of cardiovascular diseases, such as atherosclerosis, and in particular for modulating endothelial nitric oxide synthase (eNOS). The invention further relates to pharmaceutical preparations consisting of said folates and a pharmaceutically acceptable carrier, optionally in combination with other pharmaceutically active agents, as well as therapeutic methods using said folates or pharmaceutical preparations thereof.

### BACKGROUND OF THE INVENTION

Nitric oxide (NO) is an important signalling molecule. It relaxes vascular smooth muscle cells to dilate blood vessels.

It also inhibits a variety of pathological events such as activation of platelets and induction of inflammatory proteins. Loss of NO leads to high blood pressure and atherosclerotic vascular diseases.

The nitric oxide synthases (NOS) are a group of enzymes (EC 1.14.13.39) responsible for the synthesis of NO from the terminal nitrogen atom of L-arginine in the presence of O₂ and the cofactors nicotinamide adenine dinucleotide phosphate (NADPH), flavin adenine dinucleotide (FAD), flavin mononucleotide (FMN), heme, tetrahydrobiopterin (BH₄). Endothelial NOS (eNOS) generates NO in blood vessels and is involved with regulating vascular function (FIG. 9).

Production of reactive oxygen species (ROS) is increased among others in cardiovascular and pulmonary diseases. An important consequence of excessive production of ROS is oxidative inactivation of NO. For example, superoxide radical rapidly react with NO to form peroxynitrite, resulting in immediate loss of NO. ROS can oxidize BH₄, an essential cofactor for eNOS. This response leads to a condition where eNOS produces superoxide rather than NO (FIG. 10). This “uncoupling” phenomenon of eNOS likely prolongs oxidant stress.

Oxidative stress plays an important role in the pathogenesis of cardiovascular diseases, such as atherosclerosis, through effects of ROS on NO bioavailability and through interactions with numerous redox-sensitive signalling pathways. Although ROS scavenging has been proposed as a therapeutic strategy to target oxidative stress in cardiovascular diseases and in particular atherosclerosis, the outcome of clinical trials using simple “antioxidants” have been disappointing (Griendling K. K. and FitzGerald G. A., Circulation. 2003; 108:2034).

Folates are essential cofactors in one-carbon transfer reactions, and are involved in key syntheses in human, animal and vegetable cells, particularly in DNA biosynthesis and in the methylation cycle. As drugs, folates have hitherto predominantly been used as the calcium salt of 5-formyl-5,6,7,8-tetrahydrofolic acid (leucovorin) or of 5-methyl-5,6,7,8-tetrahydrofolic acid (metafolin) for the treatment of megaloblastic anaemia, as an antidote for enhancing the compatibility of folate antagonists, particularly of aminopterin and methotrexate in cancer therapy (“antifolate rescue”), for enhancing the therapeutic effect of fluorinated pyrimidines and for the treatment of auto-immune diseases such as psoriasis, for enhancing the compatibility of certain anti-parasitic substances, for instance trimethoprim-sulfamethoxazole, and for reducing the toxicity of dideazatetrahydrofolates in chemotherapy.

In addition there have been various studies relating folic acid to endothelial function, yet the results are of conflicting nature indicating that the exact effects and mechanism of folic acid and its derivatives on endothelial function is still unknown. For example, it has been suggested that plasma levels of total homocysteine (tHcy) (Wald D S et al, BMJ. 2002; 325:1202) and folate (Bunout D et al, Nutrition. 2000; 16:434) may be related with cardiovascular risk (Wald D S et al, BMJ. 2002; 325:1202) and endothelial dysfunction (Doshi S N et al, Circulation. 2002; 105:22, Hyndman M E et al, Am J Physiol Heart Circ Physiol. 2002; 282:H2167, Verhaar M C et al, Circulation. 1999; 100:335). However, studies suggesting that lowering tHcy with folic acid may retard progression of atherosclerosis were not confirmed (Lange H et al, N Engl J Med. 2004; 350:2673). In fact, recent reports on larger trials in patients with stroke (Toole J F et al, JAMA. 2004; 291:565-575) myocardial infarction (Bonaa K H et al, N Engl J Med. 2006; 354:1578) or stable coronary artery disease (CAD) (N Engl J Med. 2006; 354:1567) found that folic acid treatment did not improve clinical outcome. More recent studies have suggested that folic acid, through its circulating form 5-methyltetrahydrofolate (5-MTHF), may have antioxidant properties and exert biological effects in vascular cells that are not directly related to changes in plasma tHcy (Doshi S N et al, Arterioscler Thromb Vasc Biol. 2001; 21:1196; Doshi S N et al, Circulation. 2002; 105:22). Other previous studies have suggested that folates may have effects on NO-mediated endothelial function, such as through changes in eNOS regulation mediated by the eNOS cofactor, tetrahydrobiopterin (BH₄, Stroes E S et al, Circ Res. 2000; 86:1129; Verhaar M C et al, Circulation. 1998; 97:237; Hyndman M E et al, Am J Physiol Heart Circ Physiol. 2002; 282:H2167). In addition there have been several studies in the field of nutrition reporting beneficial effects of various vitamin preparations containing folic acid in combination with various other supplements, i.e. such as vitamin B₆, B₁₂, E and others. These preparations were typically developed for the treatment of patients with the respective nutritional deficiencies that are as a consequence at risk various illnesses, such as neuropsychiatric, vascular renal and hematologic conditions (U.S. Pat. No. 6,207,651).

Clearly, though the use of folic acid has been widespread, its mode of action remains unclear. Yet despite the above mentioned conflicting views on the mechanism and effects of folic acid, all of these studies have shown that folates by themselves seemed to have no direct effect on endothelial functions such as in vitro NO production by eNOS (Verhaar M C et al, Circulation. 1998; 97:237) and the above reported effects were exclusively observed using folates in combination with other active agents, i.e. agents which are known for their participation in eNOS coupling, such as tetrahydrobiopterin (BH₄) or a derivative thereof, which in fact is a naturally occurring cofactor of the three forms of NOS and of the aromatic amino acid hydroxylases and is involved in various biochemical reactions, and/or the amino acid arginine, which is the precursor of endogenous NO (U.S. Pat. No. 6,544,994; U.S. Pat. No. 6,995,158).

Thus there is still a lack of clear understanding in redox signalling in the vessel wall which to date prevented the development of efficient “antioxidant” therapies. Clearly, there still exists a great need for efficient therapeutic methods and agents for the prevention and/or treatment of cardiovascular diseases such as atherosclerosis.

Applicants have now surprisingly found that contrary to the state of the art folates indeed have a direct effect on specific redox mechanisms in human cardiovascular diseases such as atherosclerosis indicating that the presence of any further active agents is not necessary for the folates to develop full efficacy (naturally, if desired, one or possibly more further active agents may still be included for separate or synergistic effects). In particular it was found that folate compounds, even in the absence of any further active agents or NOS cofactors such as BH₄ or arginine, and in a concentration range that is readily achievable in vivo, rapidly improved NO-mediated endothelial function while decreasing superoxide production by preventing peroxynitrite-mediated tetrahydrobiopterin (BH₄) oxidation and reversing eNOS uncoupling, increasing vascular BH₄, BH₄/total biopterin ratio and eNOS dimer:monomer ratio, enhancing eNOS activity and furthermore possessing specific rather than general “antioxidant” effects in human atherosclerosis.

Thus folates act as a specific and effective intracellular compound useful in the prevention and/or treatment of cardiovascular diseases and in particular atherosclerosis.

### SUMMARY OF THE INVENTION

In a first aspect of the invention, new uses of folates are provided for producing a medicament containing folate as the active agent for the prevention and/or treatment of cardiovascular diseases such as atherosclerosis.

Without being bound by any specific theory, the specific therapeutic effects were achieved by modulation of endothelial nitric oxide synthase (eNOS), possibly by scavenging reactive oxygen species (ROS), such as peroxynitrite, increasing vascular BH₄ and the BH₄/total biopterin ratio, furthermore reversing eNOS uncoupling, increasing eNOS dimer:monomer ratio and direct enhancing eNOS activity. So in a concentration range that is readily achievable in vivo 5-methyltetrahydrofolate (5-MTHF) improved rapidly NO-mediated endothelial-dependent vasomotor responses and reduced vascular superoxide, both ex vivo and in vivo. These changes were not explained by direct superoxide scavenging by 5-MTHF in vitro or by changes in plasma total homocysteine in vivo. Additionally the therapeutic effects were achieved by the specific “antioxidant” effects of folates in human atherosclerosis.

In a specific embodiment the folates include pteroic acid monoglutamate (folic acid), dihydrofolic acid, 5-formyltetrahydrofolic acid, 5-methyltetrahydrofolic acid, 5,10-methylenetetrahydrofolic acid, 5,10-methenyltetrahydrofolic acid, 10-formyltetrahydrofolic acid or tetrahydrofolic acid, polyglutamates thereof, optical isomers thereof, particularly optically pure natural isomers thereof, and mixtures of optical isomers also, particularly racemic mixtures, as well as pharmaceutically acceptable salts and esters thereof and the like, optionally in combination with one or more other pharmaceutically active agents.

In another aspect of the invention, a pharmaceutical preparation is provided for the treatment and/or prevention of cardiovascular diseases, characterised in that it consists of at least one folate or a pharmaceutically acceptable salt or ester thereof and at least one pharmaceutically acceptable carrier

In another aspect of the invention, therapeutic methods are provided for the prevention and/or treatment of cardiovascular diseases, such as atherosclerosis, in particular by modulation of endothelial nitric oxide synthase (eNOS), improving NO-mediated endothelial-dependent vasomotor responses, increasing vascular BH₄ and the BH₄/total biopterin ratio, reversing eNOS uncoupling, increasing eNOS dimer:monomer ratio, direct enhancing eNOS activity and furthermore reducing vascular superoxide, possibly by scavenging reactive oxygen species (ROS), such as peroxynitrite.

In one specific embodiment the therapeutic methods comprise administering a folate, such as a pteroic acid monoglutamate (folic acid), dihydrofolic acid, 5-formyltetrahydrofolic acid, 5-methyltetrahydrofolic acid, 5,10-methylenetetrahydrofolic acid, 5,10-methenyltetrahydrofolic acid, 10-formyltetrahydrofolic acid or tetrahydrofolic acid, polyglutamates thereof, optical isomers thereof, particularly optically pure natural isomers thereof, and mixtures of optical isomers also, particularly racemic mixtures, as well as pharmaceutically acceptable salts and esters thereof or a pharmaceutical preparation consisting of at least one folate or a pharmaceutically acceptable salt or ester thereof and at least one pharmaceutically acceptable carrier, to a subject in need thereof.

Other embodiments involve administering the folate in combination with one or more other pharmaceutically active agents.

Further embodiments involve the administration routes and dosage forms of the folate or pharmaceutical preparation thereof.

Yet further embodiments may include kits or containers comprising a folate or a pharmaceutical preparation thereof optionally in combination with one or more other pharmaceutically active agents.

### BRIEF DESCRIPTION OF THE FIGURES

FIG. 1. Isometric tension studies in segments of saphenous veins from 30 patients incubated with increasing concentrations of 5-methyltetrahydrofolate (5-MTHF) for 45 minutes. The baseline relaxations were identical between the four groups and are presented as a single curve. Vessel relaxations to the endothelium-dependent agonists acetylcholine (Ach, Panel A) or bradykinin (BK, Panel B) did not change in control vessels, but increased significantly after incubation with 5-MTHF. The absolute contractions in response to phenylephrine were 7.9±0.8 g at baseline, and remained unchanged after incubation. *P<0.05, **P<0.01 vs. baseline.

FIG. 2. Superoxide and peroxynitrite production were significantly decreased after incubation with increasing concentrations of 5-methyltetrahydrofolate (5-MTHF) for 45 minutes, in both saphenous veins (SV, Panel A, n=32 and panel B, n=6) and internal mammary arteries (IMA, Panel C n=23 and Panel D n=6), compared to control vessels (incubated with buffer) from the same patients. Values expressed as median (horizontal line), 25^{th}-75^{th} percentile (box) and range (whiskers). *P<0.01 vs. control.

FIG. 3. Segments of saphenous veins obtained ˜45 minutes after intravenous infusion of 5-methyltetrahydrofoate (5-MTHF, 0.13 mg/kg body weight, n=15), had significantly greater vasomotor responses to both acetylcholine (ACh, Panel A) and bradykinin (BK, Panel B) compared to segments from patients who received placebo (n=17). There was no significant difference in vasomotor responses to nitroprusside (SNP) between patients who received intravenous 5-MTHF and those who received placebo. The absolute pre-contractions to phenylephrine in these vessels were 8.20±1.26 g and 7.72±1.31 g in the 5-MTHF and placebo-treated groups, respectively (P═NS). *P<0.05 vs. placebo.

FIG. 4. Superoxide production from intact vessel segments, was significantly lower in patients ˜45 minutes after intravenous infusion of 5-methyltetrahydrofolate (5-MTHF, 0.13 mg/kg body weight, n=15, white bars), compared to placebo patients (n=25, grey bars). Measurements were performed in paired samples of both 19 saphenous veins (SV) and internal mammary arteries (IMA) from the same patients, using 5 μM lucigeninenhanced chemiluminescence. Values expressed as median (horizontal line), 25^{th}-75^{th} percentile (box) and range (whiskers). *P<0.01 vs. placebo.

FIG. 5. Superoxide scavenging by 5-methyltetrahydrolofolate (5-MTHF) was assessed using a xanthine/xanthine oxidase system (A). 5-MTHF had a weak superoxide scavenging effect, but only at concentrations >10 μM, whereas vitamin C had the expected scavenging effect even at low concentrations. Peroxynitrite scavenging by 5-MTHF and uric acid was assessed using SIN-1 (1 μM) (B). 5-MTHF had a strong direct peroxynitrite scavenging effect at concentrations as low as 1 μM, comparable to that of equal concentrations of uric acid. Values are means±SEM of 3 separate experiments. *P<0.01 vs. vitamin C; †P<0.05 and ‡P<0.01 vs. 0 μM.

FIG. 6. Effect of intravenous infusion of 5-methyltetrahydrofolate (5-MTHF) on eNOS-derived superoxide production in saphenous veins (SV) and internal mammary arteries (IMA). Superoxide generation, measured by 5 μM lucigenin-enhanced chemiluminescence, was decreased by the NOS inhibitor L-NAME in both SV and IMA (Panel A) from patients in the placebo group (grey bars). However, L-NAME significantly increased superoxide generation in vessels ˜45 minutes after intravenous infusion of 5-MTHF (white bars). In situ detection of endothelium-derived superoxide generation in SV (Panels B and C, n=10), using dihydroethidium fluorescence, showed an increase in L-NAME-induced superoxide production from vascular endothelium (arrows) in vessels from 5-MTHF-treated patients (white bars), and a decrease in vessels from placebo-treated patients (grey bars). *P<0.05, †P<0.01, ‡P<0.001 5-MTHF vs. placebo.

FIG. 7. Tissue biopterin levels in saphenous veins (SV) and internal mammary arteries (IMA). Ex vivo incubations for 45 minutes with 5-methyltetrahydrofolate (5-MTHF; 1 μM) increased tetrahydrobiopterin (BH₄) levels (Panel A) and BH₄/total biopterin (BH₄/tBio) ratio (Panel B) compared to control, in both SV (n=20 pairs) and IMA (n=12 pairs). Similarly, vascular BH₄ (Panel C) and BH₄/tBio ratio (Panel D) were higher in 20 vessels obtained ˜45 minutes after infusion of 5-MTHF (0.13 mg/kg, n=23 for SV and n=14 for IMA), compared to vessels from patients who received placebo (n=19 for SV and n=19 for IMA). There was no significant difference in the demographic characteristics or medication between the patients in the two groups. Grey bars:control or placebo; White bars: 5-MTHF; *P<0.05; **P<0.01 vs. control or placebo. 5-MTHF (1 μM) prevented the decrease of BH₄ (Panel E) and (BH₄/tBio) ratio (Panel F), induced by the exposure of BH₄ (0.1 μM) to SIN-1 (2 μM) for 14 minutes. †p<0.05 and ‡p<0.01 vs BH₄ alone; *P<0.05 and **P<0.01 vs BH₄+SIN-1. Ex-vivo incubation of IMA (n=5 pairs) with 5-MTHF significantly increased eNOS dimer:monomer ratio as evaluated by immunoblotting (Panel G) after quantification of the eNOS band intensity (Panel H, *P<0.05 vs control).

FIG. 8. Vascular BH₄/total biopterin (BH₄/tBio) ratio was correlated with the L-NAME-induced change in superoxide production in both saphenous veins (SV, r=0.495, P=0.002) and internal mammary arteries (IMA r=0.621, P=0.001) from patients who received either placebo or 5-MTHF in vivo. Shown is the combined correlation for SV and IMA. Red open dots:placebo-treated IMA, red solid dots:5-MTHF-treated IMA; Black open dots:placebo-treated SV; Solid dots:5-MTHF-treated SV.

FIG. 9. Overview on Endothelial Nitric Oxide Synthase (eNOS) catalyzed reaction from L-arginine to NO and L-citrulline.

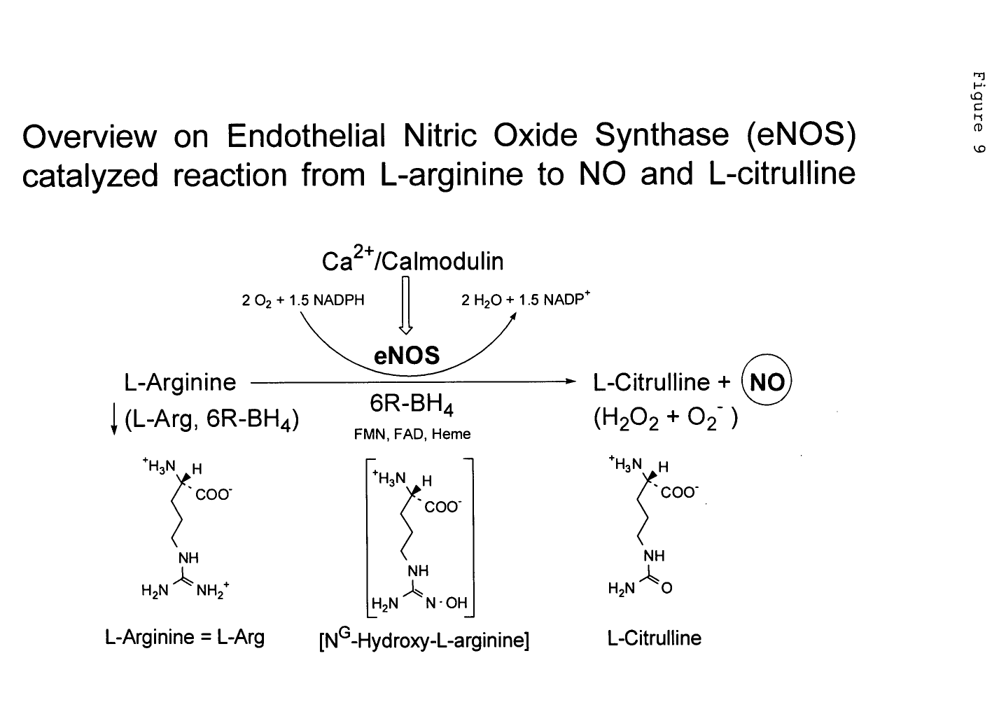

FIG. 10. NO Production by eNOS, Comparison of Physiological Pathway (eNOS Coupling) and Impaired Pathway (eNOS Uncoupling).

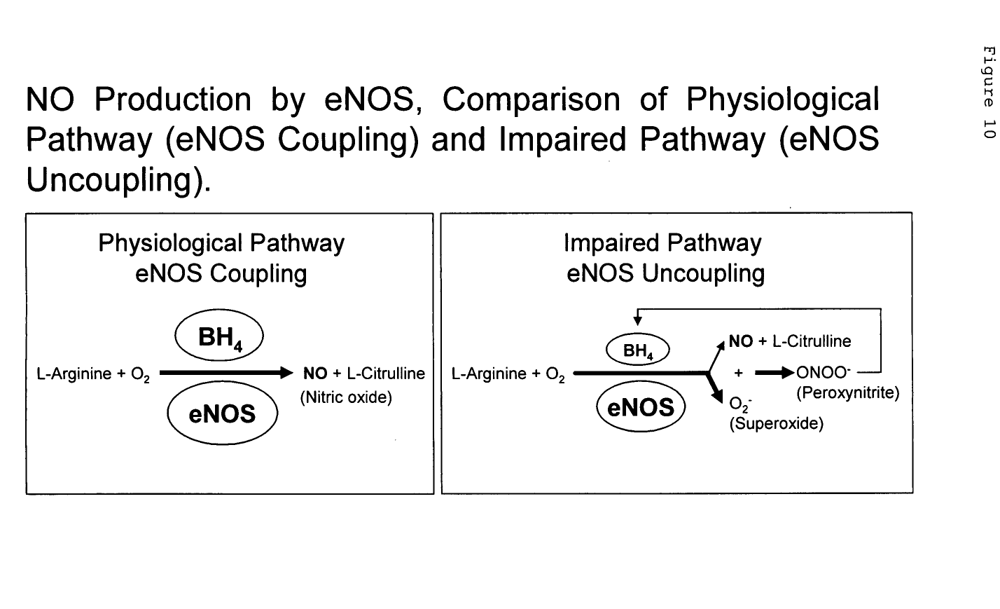

### DETAILED DESCRIPTION OF THE INVENTION

The present invention relates to the use of folates for the prevention and/or treatment of cardiovascular diseases, such as atherosclerosis, and in particular for modulating endothelial nitric oxide synthase (eNOS).

In the present text, the terms “folate” or “folate compounds” relate to both pteroic acid monoglutamate (folic acid) and to reduced forms such as dihydrofolates and tetrahydrofolates, e.g. 5-formyltetrahydrofolic acid, 5-methyltetrahydrofolic acid, 5,10-methylenetetrahydrofolic acid, 5,10-methenyltetrahydrofolic acid, 10-formyltetrahydrofolic acid and tetrahydrofolic acid, polyglutamates thereof, optical isomers thereof, particularly optically pure natural isomers thereof, and also mixtures of optical isomers also, particularly racemic mixtures, including pharmaceutically acceptable salts and esters thereof also.

Preferred folates include tetrahydrofolates, particularly natural diastereoisomeric forms of tetrahydrofolates such as 5-formyl-(6S)-tetrahydrofolic acid, 5-methyl-(6S)-tetrahydrofolic acid, 5,10-methylene-(6R)-tetrahydrofolic acid, 5,10-methenyl-(6R)-tetrahydrofolic acid, 10-formyl-(6R)-tetrahydrofolic acid, 5-formimino-(6S)-tetrahydrofolic acid or (6S)-tetrahydrofolic acid or pharmaceutically acceptable salts and esters thereof. More preferred are 5-methyl-(6S)-tetrahydrofolic acid or 5-methyl-(6R,S)-tetrahydrofolic acid, or a pharmaceutically acceptable salt or ester thereof.

In the present text, the term “pharmaceutically acceptable” as used in relation to salts and esters should include the meanings of both pharmacologically acceptable and pharmaceutically acceptable. Pharmacologically and pharmaceutically acceptable salts such as these can be alkali metal or alkaline earth metal salts, preferably sodium, potassium, magnesium or calcium salts. Pharmacologically and pharmaceutically acceptable esters such as these can be C1-C4 alkyl, C5 cycloalkyl oder C6 cycloalkyl, phenyl, C1-C4 alkylphenyl, benzyl or C1-C4-alkylbenzyl esters. The esters can be monoesters or diesters. Diesters can be homogeneous or heterogeneous. Most preferred of all are homogeneous diesters such as C1-C4 dialkylesters, for example dimethyl- or diethylesters.

In the present text, the term “cardiovascular disease” includes preferably indications caused by oxidative stress, more specifically oxidation of the endothelial cofactor tetrahydrobiopterin (BH₄) by reactive oxygen species, and includes preferably angina pectoris, coronary heart disease, hypertension, endothelial dysfunction, atherosclerosis and the like, more preferably atherosclerosis.

In the present text, the term “modulating endothelial nitric oxide synthase (eNOS)” relates to improving NO-mediated endothelial-dependent vasomotor responses and reducing vascular superoxide, both ex vivo and in vivo, by scavenging reactive oxygen species (ROS), such as peroxynitrite, increasing vascular BH₄, increasing BH₄/total biopterin ratio, reversing eNOS uncoupling, increasing eNOS dimer:monomer ratio and direct enhancing eNOS activity.

The present invention provides further a pharmaceutical preparation for the treatment and/or prevention of cardiovascular diseases, characterised in that it consists of at least one folate or a pharmaceutically acceptable salt or ester thereof and at least one pharmaceutically acceptable carrier.

In the present text, the term “pharmaceutical preparation (of the invention)” or “preparations (of the invention)” relate to enteral (e.g. oral, sublingual or rectal), parenteral or topical (e.g. transdermal) forms. Organic or inorganic substances which do not react with the active ingredient can be used as carriers, e.g. water, oil, benzyl alcohol, polyethylene glycol, glycerol triacetate or other fatty acid glycerides, gelatine, lecithin, cyclodextrin, carbohydrates such as lactobiose or starch, magnesium stearate, talc or cellulose. Tablets, drágees, capsules, powder, syrup, concentrates or drops are preferably used for oral application, suppositories are preferably used for rectal application, and water- or oil-based solutions or lyophilisates are preferably used for parenteral application.

Suspensions, emulsions or implants can also be used, and patches or creams can be used for topical application.

Preparations for parenteral application comprise sterile aqueous and nonaqueous injection solutions of the active compounds, which are preferably isotonic with the blood of the recipient.

These preparations may further comprise stabilisers, additives for the controlled release of the pharmaceutically active compound, antioxidants, buffers, bacteriostatic agents and adjuvants for obtaining an isotonic solution. Aqueous and nonaqueous sterile suspensions can comprise suspension additives and thickeners.

The preparations of the invention can exist as a single dose container or as a multiple dose container, e.g. as welded ampoules; it can be stored as a freeze-dried (lyophilised) product and when needed can be prepared for use by adding a sterile liquid, for example water or salt solution. Sterile powders, granules or tablets can be used similarly.

Despite most preferred pharmaceutical preparations contain only folates or a pharmaceutically acceptable salt or ester thereof as the active agent, all the preparations of the present invention may comprise in addition one or more other pharmaceutically active compounds which act separately or synergistically with the preparation of the invention. When comprising in addition to folates another pharmaceutically active compound then preferred preparations comprise only one more pharmaceutically active compound. In particular, these are substances which are directly involved in the folate cycle or which influence the folate cycle or which have an additional anti-inflammatory effect, such as vitamins, antioxidants, radical scavengers, biopterins, lipid reducers, immuno-suppressive agents, non-steroidal anti-inflammatory substances and/or other active ingredients.

Preferred vitamins include vitamin B₂, B₆, B₁₂ or ascorbic acid, glutathione, acetylcysteine, betaine.

Preferred biopterins include biopterins in all stages of oxidation, and isomeric forms of biopterin, especially L-erythro-biopterin, 7,8-dihydrobiopterin and 5,6,7,8-tetrahydrobiopterin, particularly L-sepiapterin, D-neopterin, xanthopterin and 6-hydroxymethyl-pterin.

Preferred antioxidants include vitamin E or beta carotene.

Preferred lipid reducers include clofibric acid derivatives (fibrates), e.g. clofibrate, bezafibrate, etofibrate, fenofibrate, gemfibrozil, ion exchange resins e.g. colestyramine or colestipol, nicotinic acid (and derivatives thereof), e.g. acipimox, sitosterin, HMG-CoA-reductase inhibitors, e.g. atorvastatin, lovastatin, pravastatin, simvastatin, fluvastatin, rosuvastatin or cerivastatin and cholesterol absorption inhibitors, e.g. ezetimib.

Preferred immuno-suppressive agents include corticosteroids, mycophenolates, mofetil, rapamycin, calcineurin inhibitors, mono- and polyclonal antibodies, and growth factors such as erythropoetin or GM-CSF.

Preferred non-steroidal anti-inflammatory substances include pentoxyfyllin, sulfasalazin, gold, aspirin, omega-3 fatty acids, thrombocyte aggregation inhibitors such as glycoprotein IIb/IIIa receptor inhibitors, hormones, flavinoids or other non-steroidal anti-inflammatory carboxylic acids such as aspirin, salsalate, diflunisal or choline magnesium trisalicylic acid, or other non-steroidal anti-inflammatory propionic acids such as ibuprofen, naproxen, fenoprofen, ketoprofen, flurbiprofen or oxaprozin, or other non-steroidal anti-inflammatory acetic acid derivatives such as indomethacin, tolmetin, sulindac, diclofenac or etodolac, or other non-steroidal anti-inflammatory fenamates such as meclofenamate or mefenamic acid, or other non-steroidal anti-inflammatory enolic acid derivatives such as piroxicam or phenylbutazone, or other non-steroidal anti-inflammatory naphthylkanones such as nabumetone, as well as COX-2 inhibitors such as celecoxib or rofecoxib. This class of substances also includes substances with an anti-inflammatory effect such as beta-blockers, anti-cytokine antibodies e.g. anti-TNF-alpha antibody, or perfusion solutions for organ preservation such as Eurocollins, HTK or University of Wisconsin (UW) solution.

Most preferred preparations when comprising in addition to folates one or more other pharmaceutically active compounds, are pharmaceutical preparations consisting of at least one folate or a pharmaceutically acceptable salt or ester thereof in combination with aspirin or ascorbic acid and at least one pharmaceutically acceptable carrier.

The present invention also provides methods for the treatment and/or prevention of cardiovascular diseases, comprising administering a therapeutically effective amount of at least one folate or a pharmaceutically acceptable salt or ester thereof, or a pharmaceutical preparation consisting of at least one folate or a pharmaceutically acceptable salt or ester thereof and at least one pharmaceutically acceptable carrier to a subject in need of such treatment and/or prevention.

The preparation comprises between 0.001 mg and 1,000 mg of the active ingredient per dose. For prevention, preparations are used which preferably contain between 5 μg and 1,000 μg of the active ingredient per dose. For therapy, preparations are used which preferably contain between 0.1 mg and 200 mg of the active ingredient per dose. The dosage depends on the form of therapy, on the form of application of the preparation, the administration route, and on the age, weight, nutrition and state of the patient. Treatment can commence with a lower dosage below the optimum amount and can be increased in order to achieve the optimum effect. The dosages used in prevention preferably range between 5 μg and 5,000 μg per day, particularly between 100 μg and 1,000 μg per day. The optimum dosages in therapy range between 0.1 mg and 100 mg per day, particularly between 0.5 mg and 5 mg per day. Administration can be effected either as a single administration or as a repeated dose.

Based on the preceding description, the person skilled in this field can immediately deduce the crucial elements of the invention, and, without departing from the basic idea and scope of the invention, can make changes and additions and can thereby adapt the invention to different requirements and conditions.

The entire disclosure of all patent applications, patents and publications which are cited in this text are included by reference thereto. The following examples can be carried out with similar success by replacing the generic or specifically described products and/or process conditions of this invention by those which are given in the following examples. The following specific embodiments are also purely exemplary, and should by no means be considered as limiting the remainder of the disclosure.

### EXAMPLES

### Methods

Patients. 117 patients with coronary artery disease undergoing routine coronary artery bypass grafting surgery (CABG) were studied at the John Radcliffe Hospital, Oxford, UK (61 included in the ex vivo and 56 in the in vivo part of the study). Exclusion criteria were the existence of any inflammatory, infective, liver or renal disease or malignancy. Patients receiving non-steroidal anti-inflammatory drugs, as well as any dietary supplement (such as folic acid or antioxidant vitamins) were also excluded. There were no significant differences in demographic characteristics or baseline plasma tHcy between the treatment groups (data not shown). The study protocol was approved by the local Research Ethics Committee, and each patient gave written informed consent.

Vessel Harvesting and Ex Vivo Studies. Samples of SV (n=38) and IMA (n=46) from a total of 61 patients, were harvested during CABG operation, as previously described (Channon K M, Trends Cardiovasc Med 2004; 14:323-327; Guzik T J et al., Circulation 2002; 105:1656-1662). Vessel segments were transferred to the laboratory within 30 minutes, in ice cold Krebs-Henseleit buffer. Segments of SV and IMA were incubated with 5-MTHF (0-100 μM) in Krebs-Henseleit buffer for 45 minutes prior to assays of endothelial function, superoxide/peroxynitrite production, radio-labelled arginine/citrulline conversion, western blotting or tissue 5-MTHF and biopterin levels.

In Vivo Studies. Patients (n=56) undergoing CABG participated in a double-blind placebo controlled study, where they received an intravenous infusion of either 5-MTHF (Merck Eprova, Switzerland) or placebo, administered before the CABG. 5-MTHF was administered at a dose of 0.13 mg/kg body weight, that in preliminary studies achieved a plasma concentration ˜2-3 μM immediately after administration, and 1-2 μM at 45 minutes after infusion. This concentration was chosen based on the dose-response analysis performed in the ex vivo experiments. Samples 5 of SV and IMA were harvested ˜45 minutes after the infusion of 5-MTHF (mean time of harvesting 45.7±2.9 minutes after infusion), and assayed for NO-mediated vasomotor function, vascular superoxide production, tissue 5-MTHF and biopterin levels, as described below.

Vasomotor Studies. Endothelium-dependent and endothelium-independent dilatation were assessed using isometric tension studies, as previously described (Guzik T J et al, Circ Res. 2000; 86:e85). Vessel rings were equilibrated and passively pre-tensioned to 3 g, an optimal resting tension that was determined in baseline studies of contractile response to KCl. Following precontraction with phenylephrine (3×10⁻⁶M), vasomotor responses to the endothelium-mediated agonists acetylcholine (ACh, 10⁻⁹M to 10⁻⁵M) and bradykinin (BK, 10⁻⁹ M to 10⁻⁵M), were quantified in 4 equally sized segments from the same vessel. In the ex vivo experiments, vasomotor responses were repeated after incubation for 45 minutes with 0 (control), 1, 10 or 100 μM 5-MTHF, added to the organ bath chambers. Finally, relaxations to the NO donor sodium nitroprusside (SNP, 10⁻¹⁰M to 10⁻⁶M), were evaluated in the presence of the NOS inhibitor NG-nitro-L-arginine methyl ester (L-NAME; 100 μM).

Superoxide/Peroxynitrite Measurements. Vascular superoxide production was measured from fresh human vessels using lucigenin (5 μM)-enhanced chemiluminescence as previously described (Skatchkov M P et al, Biochem Biophys Res Commun. 1999; 254:319). Samples of SV and IMA from the same patient were opened longitudinally to expose the endothelial surface and then equilibrated for 20 minutes in oxygenated (95% O₂/5% CO₂) Krebs-HEPES buffer (2 mL, pH=7.4) at 37° C. 14. NOS-derived superoxide was measured by adding L-NAME (100 μM) in the equilibrating buffer, and by calculating the difference in superoxide signal compared with basal conditions. Vascular peroxynitrite was determined using luminol (100 μM) instead of lucigenin, and by subtracting the remaining signal after adding the specific peroxynitrite scavenger uric acid (1 mM), as previously described (Laursen J B et al, Circulation. 2001; 103:1282).

Oxidative Fluorescent Microtopography. In situ superoxide production was determined in vessel cryosections with the oxidative fluorescent dye dihydroethidium (DHE) (Bendall J K et al, Circ Res. 2005; 97:864). Paired cryosections (30 μM) from the same vessel were incubated with DHE (2 μmol/L) in PBS, with or without L-NAME (100 μM). Fluorescence images (x40, Zeiss LSM 510 META laser 6 scanning confocal microscope) were obtained from each vessel quadrant, incorporating the luminal side of the vessel to quantify endothelial cell fluorescence. In each case, segments of vessel rings (with and without L-NAME) were analyzed in parallel with identical imaging parameters. DHE image analysis was carried out in a blinded fashion, and by two independent investigators, by using Image-Pro Plus software (Media Cybernetics).

Superoxide and Peroxynitrite Scavenging Assays. The direct superoxide scavenging effect of 5-MTHF was evaluated with the use of xanthine/xanthine oxidase system. Superoxide production was induced by adding xanthine oxidase (6.67 MU/mL) in Krebs HEPES buffer (2 mL, pH=7.4) at 37° C., containing xanthine (0.133 mM) and lucigenin (5 μM), in the presence of 5-MTHF (0-100 μM) or equal concentrations of ascorbic acid as positive control. Peroxynitrite production was induced by adding 3-morpholinosydnonimine (SIN-1, 1 μM) in Krebs HEPES buffer (2 mL, pH=7.4) at 37° C. containing luminol (100 μM), in the presence of different concentrations of 5-MTHF (0-100 μM) or equal concentrations of uric acid as positive control as previously described (Kuzkaya N et al, J Biol Chem. 2003; 278:22546). The scavenging effects of 5-MTHF, vitamin C or uric acid were calculated as the percent inhibition of chemiluminescence compared with control.

Determination of 5-MTHF and tHcy. Blood samples were collected prior to administration of 5-MTHF or placebo, and at the time of vessel harvesting. Plasma total homocysteine (tHcy) was measured by a fluorescence polarization immunoassay adapted to the IMx analyser (Abbot Diagnostics). Plasma and tissue levels of 5-MTHF were determined by high performance liquid chromatography (HPLC), as previously described (Leeming R J et al, Metabolism. 1990; 39:902).

Determination of Biopterin Levels. Biopterin levels in human vessels were determined by HPLC, as previously described (Alp N J et al, J Clin Invest. 2003; 112:725) and expressed as pmol/g of tissue. To examine the effect of 5-MTHF on peroxynitrite-induced oxidation of BH₄, SIN-1 (2 μM) was used to oxidize BH₄ (0.1 μM) in the presence or absence of 5-MTHF (1 μM) at 37° C. for 14 minutes (Milstien S and Katusic Z. Biochem Biophys Res Commun. 1999; 263:681).

Evaluation of eNOS Activity in Intact Vessels. The activity of eNOS was estimated by HPLC quantification of radio-labelled arginine to citrulline conversion from intact vessel rings, as previously described (de Bono J et al, Nitric Oxide. 2006; (in Press)).

Western Blots analysis. The eNOS dimer:monomer ratio in paired IMA samples incubated for 45 minutes in the presence or absence of 5-MTHF 1 μM was measured by western blotting as described previously (Cai S et al, Cardiovasc Res. 2002; 55:838). Bands were visualized using chemiluminescence, and quantified using NIH Image software.

Statistical Analysis. Analyses were performed using the SPSS 12.0 statistical package for Windows (SPSS Inc, Illinois, USA). Normally-distributed data are presented as mean±SEM, while non-normally distributed variables (such as vascular superoxide) are presented as median (25^{th}-75^{th} percentiles values). Baseline comparisons between groups were performed with one-way ANOVA for multiple comparisons, followed by Bonferoni correction. The effects of 5-MTHF on vasomotor responses in each vessel ring were assessed by two-way ANOVA for repeated measurements. The effects of 5-MTHF incubations on superoxide production and levels of 5-MTHF or tHcy were assessed by Mann-Whitney U tests, Wilcoxon rank tests or t-tests for unpaired or paired data, as appropriate. A two-tailed P<0.05 was considered as statistically significant.

### Example 1

### Effects of 5-MTHF on Endothelial Function and on Superoxide and Peroxynitrite Production in Human Vessels Ex Vivo

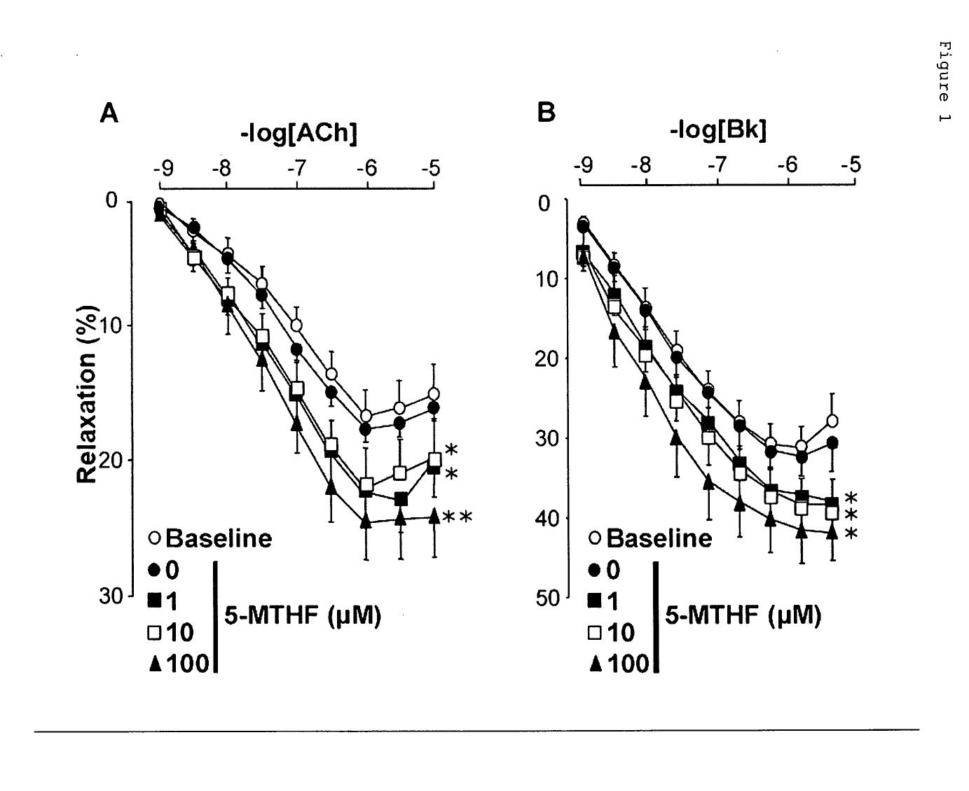

The effects of 5-MTHF on vasomotor responses to ACh and BK in vessel segments at baseline and after 45-minutes incubation in organ chambers with 0-100 μM 5-MTHF were investigated. Relaxations in response to acetylcholine (Ach) or bradykinin (BK) were similar between the 4 rings from the same vessel at baseline. FIG. 1 shows that maximum relaxation responses to Ach (Panel A) or BK (Panel B) were significantly increased after 45 minutes of incubation with 5-MTHF 1 μM, but remained unchanged in the control segments. The absolute contractions in response to phenylephrine were 7.9±0.8 g at baseline, and remained unchanged after incubation (*P<0.05, **P<0.01 vs. baseline). Higher concentrations of 5-MTHF (10 or 100 μM) resulted in no further increase in maximum relaxations to either Ach or BK. The maximum relaxations to ACh were significantly correlated with the respective relaxations to BK in the same patients (r=0.746, P<0.001). Vascular 5-MTHF levels confirmed dose-dependent increases with increasing concentration of 5-MTHF incubation, from 0.40±0.06 nmol/g tissue in control, to 3.40±0.71, 21.3±8.10 and 57.2±18.9 nmol/g tissue after incubation of 25 SV with 5-MTHF 1, 10 and 100 μM respectively (P<0.05 for all vs. control). There were no differences in the vasomotor responses to the endothelium-independent agonist, SNP, between control vessel segments and those incubated with 5-MTHF (data not shown), indicating a specific effect of 5-MTHF on NO-mediated endothelial function.

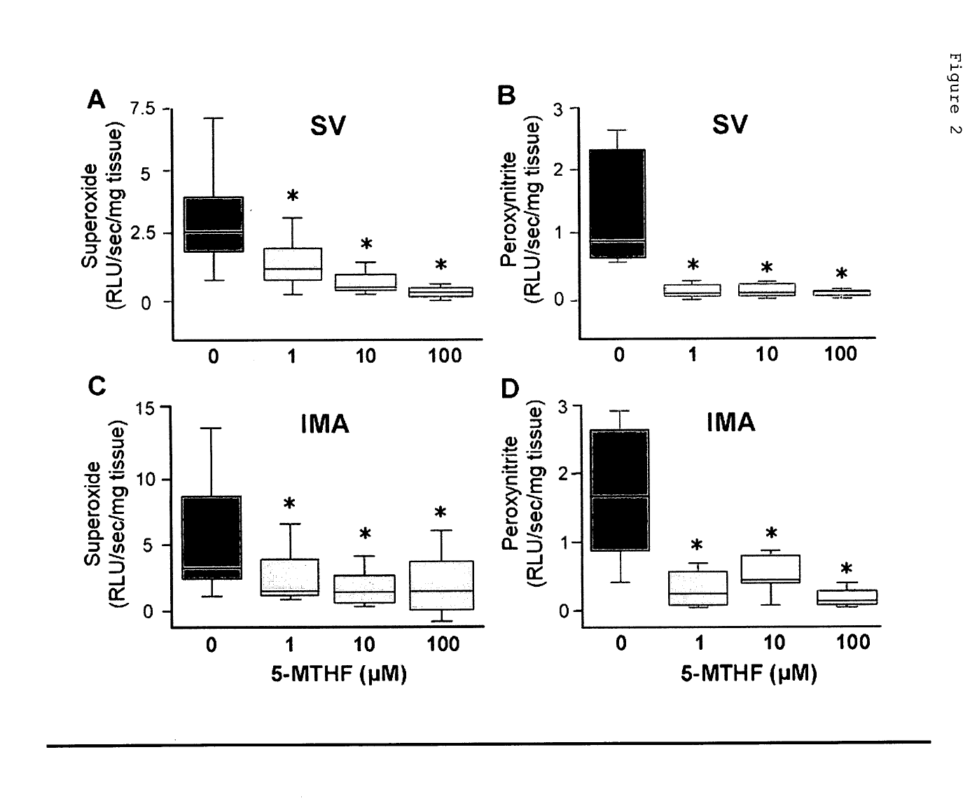

Vascular superoxide and peroxynitrite production from SV and IMA were determined after 45 minutes incubation with either buffer alone or with 5-MTHF at 1, 10 or 100 μM. FIG. 2 shows that superoxide and peroxynitrite production were significantly decreased after incubation with increasing concentrations of 5-methyltetrahydrofolate (5-MTHF) for 45 minutes, in both saphenous veins (SV, Panel A, n=32 and panel B, n=6) and internal mammary arteries (IMA, Panel C n=23 and Panel D n=6), compared to control vessels (incubated with buffer) from the same patients (Values expressed as median (horizontal line), 25^{th}-75^{th} percentile (box) and range (whiskers); *P<0.01 vs. control). Thus, incubation with 1 μM 5-MTHF significantly reduced both vascular superoxide and peroxynitrite production in SV and IMA, with no further reductions at higher 5-MTHF concentrations.

### Example 2

### Effects of 5-MTHF on Endothelial Function and Superoxide Production in Human Vessels In Vivo

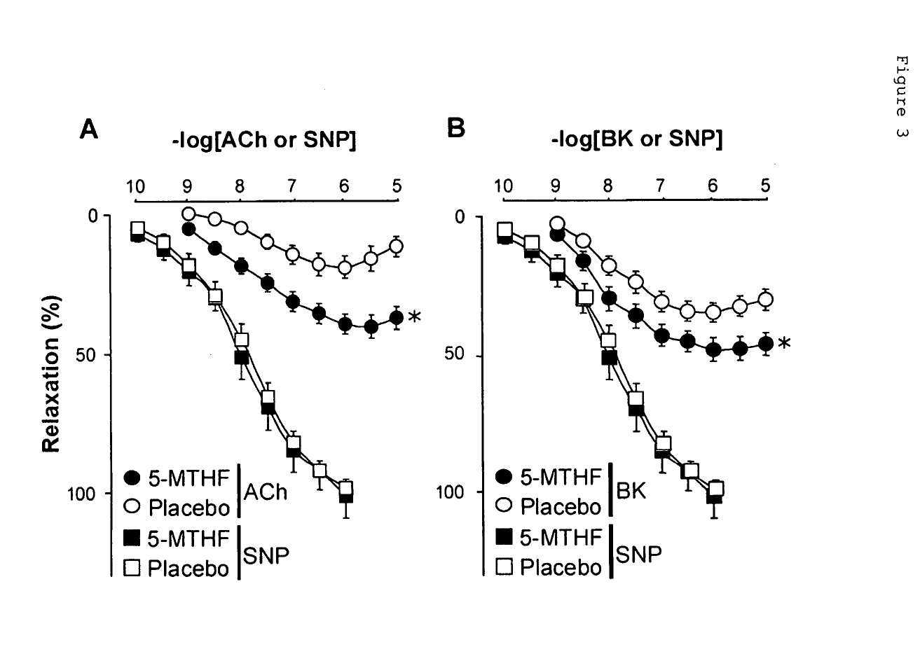

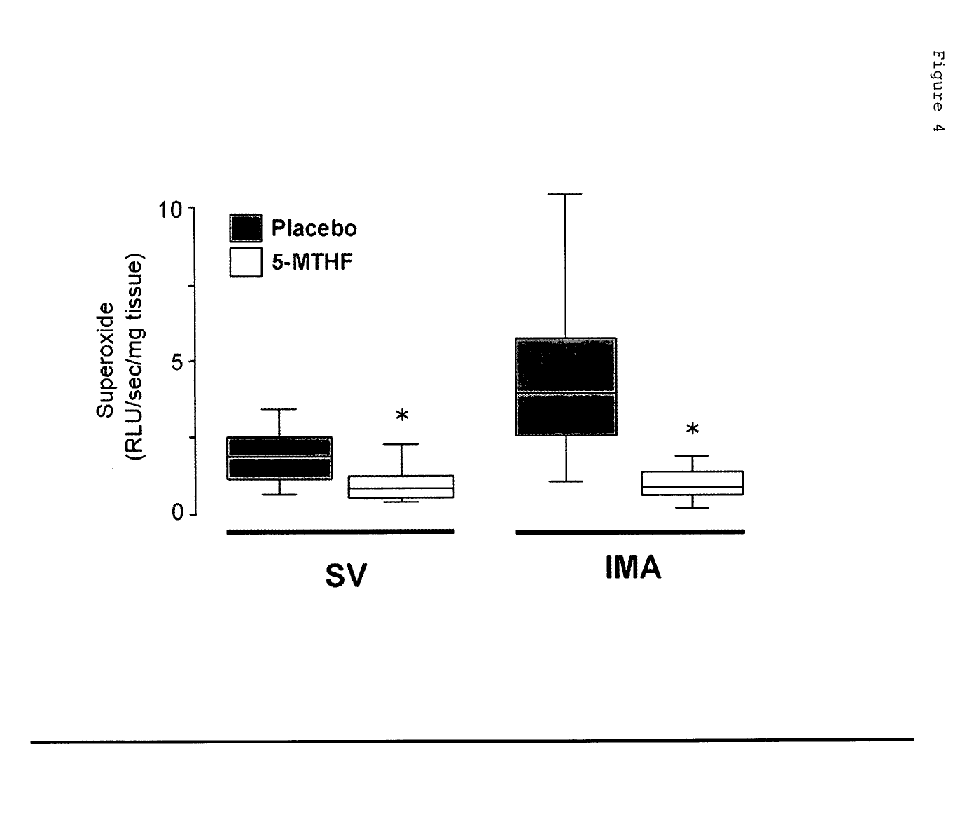

Patients were randomized to receive either i.v. 5-MTHF or placebo in a double-blind fashion. Baseline plasma levels of 5-MTHF were not significantly different between patients who received intravenous 5-MTHF (32.4∓9.01 nM) compared with placebo (26.4±4.68 nM, P═NS). In contrast, plasma 5-MTHF levels were significantly increased at the time of vessel harvesting in the 5-MTHF-treated group (2.28±0.21 μM; P<0.001 vs. baseline) but remained unchanged in the placebo-treated group (25.8±6.85 nM, P═NS vs. baseline, P<0.001 vs. 5-MTHF-treated group). Correspondingly, tissue 5-MTHF levels in SV (9.35±1.72 nmol/g) and IMA (12.7±2.58 nmol/g) were significantly increased in the 5-MTHF treated group compared to placebo group (1.19±0.25 nmol/g for SV and 1.54±0.38 nmol/g for IMA respectively, P<0.001 for both), confirming that intravenous administration of 5-MTHF substantially increases vascular tissue levels. Plasma tHcy was modestly decreased in patients who received 5-MTHF (from 11.0±0.62 to 9.42±0.46 μmol/L, P<0.05) 45 minutes after infusion, but remained unchanged in the placebo group (from 12.0±1.23 to 11.6±1.26 μmol/L, P═NS). The effects of in vivo administration of 5-MTHF on endothelial function was then evaluated by quantification of vasomotor responses to ACh and BK in vessel segments from patients who received either placebo or 5-MTHF. FIG. 3 indicates that intravenous 5-MTHF had striking effects on maximal vasorelaxations to both ACh (Panel A, 43.2±4.5%) and BK (Panel B, 51.4±4.2%) compared with the placebo group (19.4±3.3% and 37.1±3.7% respectively, P<0.05 for both), whereas endothelium-independent vasomotor responses to nitroprusside (SNP) were identical between the two groups (FIG. 3). The absolute pre-contractions to phenylephrine in these vessels were 8.20±1.26 g and 7.72±1.31 g in the 5-MTHF and placebo-treated groups, respectively (P═NS) (*P<0.05 vs. placebo). There was no correlation between maximal relaxations to either ACh or BK and plasma tHcy (Ach r=0.272, P=0.114; BK r=−0.090, P=0.605). Basal superoxide production was measured in paired segments of SV and IMA from 40 patients (25 placebo and 15 who received intravenous 5-MTHF). Vascular superoxide was significantly lower in both SV and IMA in the intravenous 5-MTHF group (ca. 45 minutes after intraveneous infusion of 5-MTHF, 0.13 mg/kg body weight, n=15, white bars), compared to placebo group (n=25, grey bars; P<0.01 for both, FIG. 4). Measurements were performed in paired samples of both saphenous veins (SV) and internal mammary arteries (IMA) from the same patients, using 5 μM lucigenin enhanced chemiluminescence. Values expressed as median (horizontal line), 25^{th}-75^{th} percentile (box) and range (whiskers). *P<0.01 vs. placebo.

### Example 3

### Direct Superoxide/Peroxynitrite Scavenging Capacity of 5-MTHF

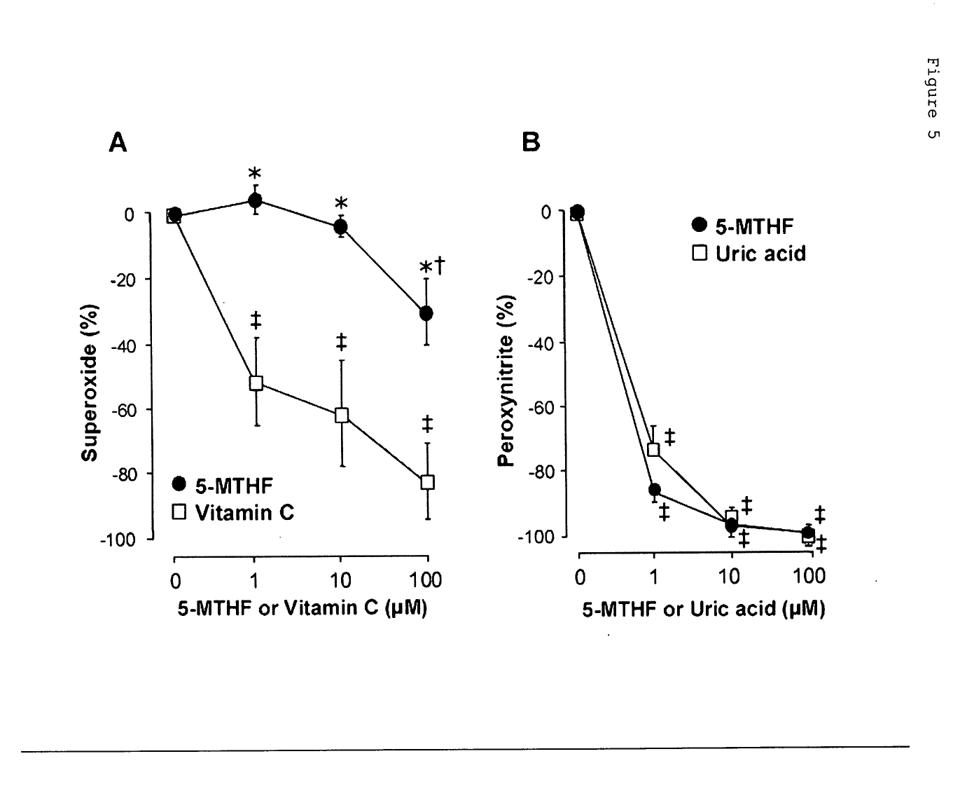

In order to investigate whether direct superoxide scavenging by 5-MTHF could account for its effects on vascular superoxide production and endothelial function, the ability of 5-MTHF to scavenge superoxide was assessed and compared with the known superoxide scavenger ascorbic acid (vitamin C). When added to a xanthine/xanthine oxidase system generating superoxide at similar levels to those observed in vascular tissues, ascorbic acid had a potent scavenging effect, reducing measurable superoxide by 50% at 1 μM, whereas low concentrations of 5-MTHF (1-10 μM) had no detectable effect on measurable superoxide (FIG. 5), with only modest superoxide scavenging observed even at very high 5-MTHF concentrations (100 μM).

The direct peroxynitrite scavenging capacity of 5-MTHF was assessed in comparison with the known peroxynitrite scavenger uric acid. FIG. 5 shows that when added to a SIN-1 system, 5-MTHF had a potent scavenging effect comparable to the effect of uric acid at the same concentration, whereby measurable peroxynitrite was reduced by 75% at 1 μM (FIG. 5), suggesting that 5-MTHF, at concentrations used in the in vivo studies, exerted a significant peroxynitrite-scavenging effect (values are means±SEM of 3 separate experiments. *P<0.01 vs. vitamin C; †P<0.05 and ‡P<0.01 vs. 0 μM).

### Example 4

### Effects of 5-MTHF on eNOS-Derived Vascular Superoxide Production and eNOS Activity

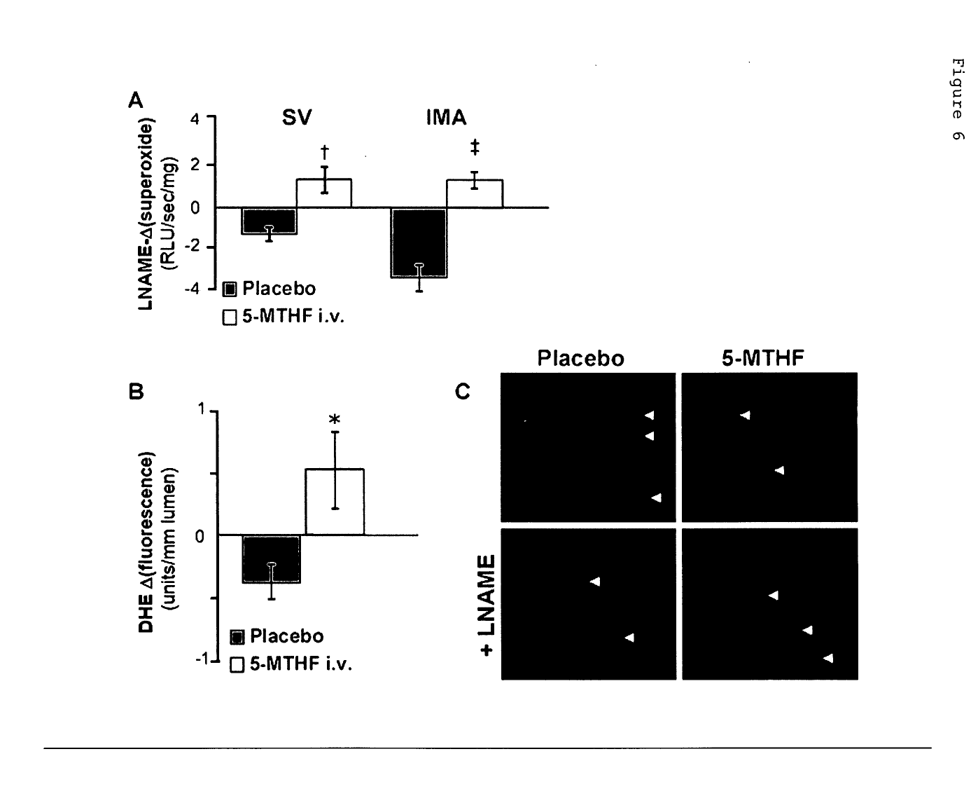

NOS-derived superoxide production was estimated in paired samples of SV and IMA, by quantifying the effects of NOS inhibition, using L-NAME. Vascular superoxide production was decreased by L-NAME in both SV and IMA in the placebo group, suggesting a net contribution to vascular superoxide production by NOS. In contrast, L-NAME increased superoxide production in vessels from 5-MTHF treated patients, suggesting net NO production (FIG. 6). Importantly, L-NAME inhibitable superoxide production in SV or IMA was correlated with plasma 5-MTHF (r=0.511, p=0.006 and r=0.690, p=0.0001 respectively) but not with plasma tHcy levels (r=0.40, p=0.829 and r=−0.286, p=0.106 respectively).

Oxidative fluorescent microtopography with DHE was used to visualize vascular superoxide production and to specify the changes in eNOS-mediated endothelial superoxide production (FIG. 6). L-NAME decreased endothelial DHE fluorescence in vessels from placebo patients (indicating eNOS uncoupling), whereas LNAME increased endothelial DHE fluorescence in vessels from 5-MTHF-treated patients, suggesting an improvement of eNOS coupling in these vessels (FIG. 6). Importantly, DHE fluorescence in other regions of the vessel wall was unaffected by 5-MTHF, providing a within-section control and demonstrating the endothelium-specific effect of NOS inhibition. HPLC analysis was used to further evaluate the effects of 5-MTHF on eNOS-activity, by measureing the conversion of radio-labelled arginine to citrulline in samples of IMA from 12 patients, incubated with or without 5-MTHF 1 μM for 45 minutes. A significant increase in citrulline production in vessels incubated with 5-MTHF (0.20±0.03%/g tissue) was observed compared to paired control vessels from the same patients (0.14±0.02%/g, p<0.05), suggesting that 5-MTHF directly increases eNOS activity in human vessels. Thus, 5-MTHF reduced vascular superoxide production and improved NO-mediated endothelial function through effects on eNOS coupling.

### Example 5

### Effects of 5-MTHF on Tetrahydrobiopterin Levels and eNOS Dimer:Monomer Ratio in Human Vessels

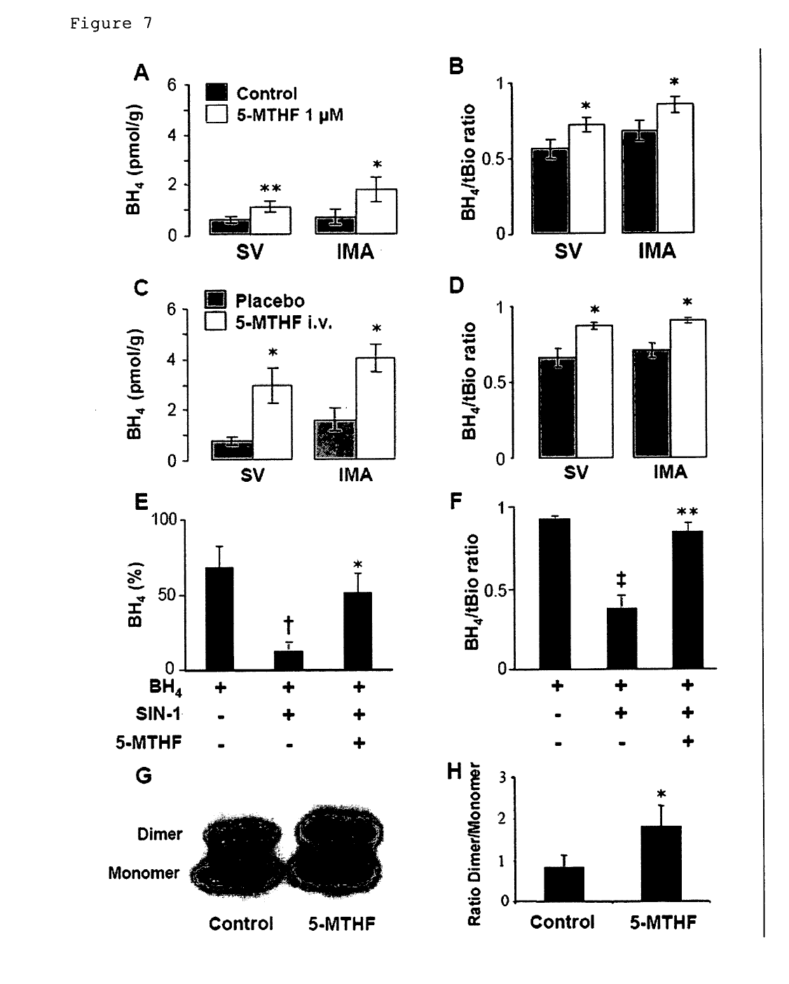

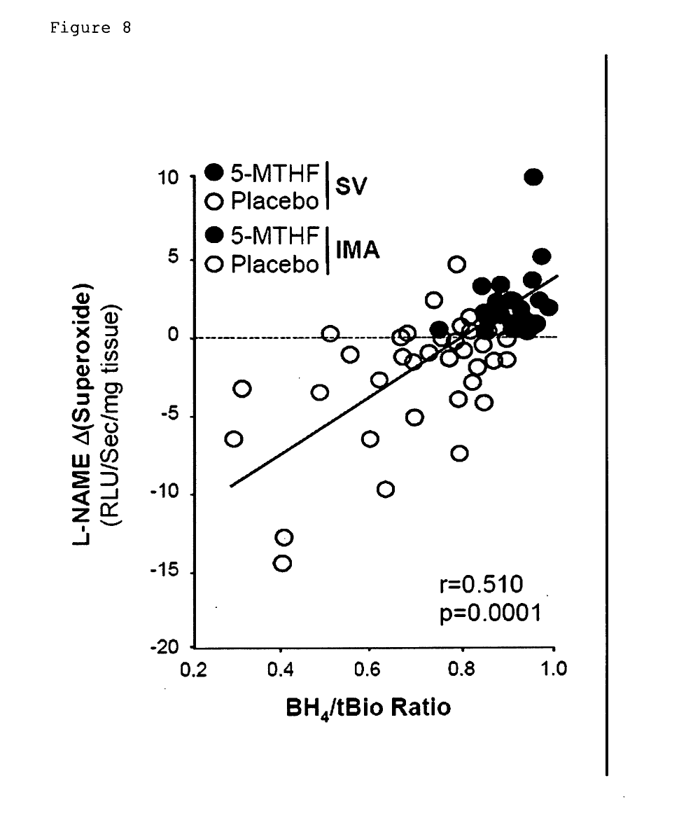

FIG. 7 shows that incubation of SV for 45 minutes ex vivo with 1 μM 5-MTHF significantly increased vascular BH₄ as well as the BH₄/tBio ratio. More strikingly, both the absolute levels of BH₄ and the BH₄/tBio ratio were significantly elevated in patients who received intravenous infusion of 5-MTHF in vivo, compared to placebo-treated patients (FIG. 7). FIG. 7 also indicates that 5-MTHF also significantly reduced the decrease of both BH₄ and BH₄/tBio ratio after exposure to peroxynitrite generated by SIN-1, which is in accordance with the previous finding that 5-MTHF is a strong peroxynitrite scavenger. Furthermore, the dimer:monomer ratio was also increased after exposure of IMA rings to 5-MTHF 1 μM (FIG. 7). It was further found that vascular BH₄/tBio ratio was significantly correlated with the LNAME-induced change in superoxide production FIG. 8 shows the combined correlation for SV and IMA (red open dots:placebo-treated IMA, red solid dots:5-MTHF-treated IMA; Black open dots:placebo-treated SV; Solid dots: 5-MTHF-treated SV.) in both SV (r=0.495, P=0.002) and IMA (r=0.621, P=0.001) from individual patients. Finally, BH₄/tBio ratio in SV and IMA was significantly correlated with plasma 5-MTHF (r=0.498, p=0.01 and r=0.656, p=0.0001 respectively) but not with plasma tHcy (r=0.033, p=0.836 and r=−0.165, p=0.374 respectively), suggesting that 5-MTHF itself rather than plasma tHcy is the critical parameter preventing the oxidation of BH₄ in human vessels in vivo. These observations suggest a direct functional relationship between vascular BH₄ availability and eNOS coupling in human vessels that is a major determinant of both NO-mediated endothelial function and vascular superoxide production.

1. Use of a folate for producing a medicament containing folate as the active agent for the prevention and/or treatment of cardiovascular diseases.

2. Use according to claim 1 characterized in that the cardiovascular disease is atherosclerosis.

3. Use according to claim 1, characterised in that the folate is selected from the group consisting of pteroic acid monoglutamate (folic acid), dihydrofolic acid, 5-formyltetrahydrofolic acid, 5-methyltetrahydrofolic acid, 5,10-methylenetetrahydrofolic acid, 5,10-methenyltetrahydrofolic acid, 10-formyltetrahydrofolic acid or tetrahydrofolic acid, polyglutamates thereof, optical isomers thereof, particularly optically pure natural isomers thereof, and mixtures of optical isomers also, particularly racemic mixtures, as well as pharmaceutically acceptable salts and esters thereof.

4. Use according to claim 1, characterised in that the folate is selected from 5-methyl-(6S)-tetrahydrofolic acid, 5-methyl-(6R)-tetrahydrofolic acid, 5-methyl-(6R,S)-tetrahydrofolic acid, 5-formyl-(6S)-tetrahydrofolic acid, 5-formyl-(6R)-tetrahydrofolic acid or 5-formyl-(6R,S)-tetrahydrofolic acid, or a pharmaceutically acceptable salt or ester thereof.

5. Use according to claim 1, characterised in that the folate is selected from 5-methyl-(6S)-tetrahydrofolic acid or 5-methyl-(6R,S)-tetrahydrofolic acid, or a pharmaceutically acceptable salt or ester thereof.

6. A pharmaceutical preparation for the treatment and/or prevention of cardiovascular diseases, characterised in that it consists of at least one folate or a pharmaceutically acceptable salt or ester thereof and at least one pharmaceutically acceptable carrier.

7. The pharmaceutical preparation according to claim 6, characterised in that the folate is selected from the group consisting of pteroic acid monoglutamate (folic acid), dihydrofolic acid, 5-formyltetrahydrofolic acid, 5-methyltetrahydrofolic acid, 5,10-methylenetetrahydrofolic acid, 5,10-methenyltetrahydrofolic acid, 10-formyltetrahydrofolic acid or tetrahydrofolic acid, polyglutamates thereof, optical isomers thereof, particularly optically pure natural isomers thereof, and mixtures of optical isomers also, particularly racemic mixtures, as well as pharmaceutically acceptable salts and esters thereof.

8. The pharmaceutical preparation according to claim 6, characterised in that it comprises one or more additional pharmaceutically acceptable active ingredients and/or adjuvants.

9. The pharmaceutical preparation according to claim 8, wherein the pharmaceutically acceptable active ingredient is aspirin.

10. The pharmaceutical preparation according to claim 8, wherein the pharmaceutically acceptable active ingredient is ascorbic acid.

11. Method for the treatment and/or prevention of cardiovascular diseases, comprising administering a therapeutically effective amount of at least one folate or a pharmaceutically acceptable salt or ester thereof or a pharmaceutical preparation consisting of at least one folate or a pharmaceutically acceptable salt or ester thereof and at least one pharmaceutically acceptable carrier to a subject in need of such treatment and/or prevention.

12. Method according to claim 11, wherein the cardiovascular disease is atherosclerosis.

13. Method according to claim 11, wherein the at least one folate is selected from the group consisting of pteroic acid monoglutamate (folic acid), dihydrofolic acid, 5-formyltetrahydrofolic acid, 5-methyltetrahydrofolic acid, 5,10-methylenetetrahydrofolic acid, 5,10-methenyltetrahydrofolic acid, 10-formyltetrahydrofolic acid or tetrahydrofolic acid, polyglutamates thereof, optical isomers thereof, particularly optically pure natural isomers thereof, and mixtures of optical isomers also, particularly racemic mixtures, as well as pharmaceutically acceptable salts and esters thereof.

14. Method according to claim 11, wherein the at least one folate is selected from 5-methyl-(6S)-tetrahydrofolic acid, 5-methyl-(6R)-tetrahydrofolic acid, 5-methyl-(6R,S)-tetrahydrofolic acid, 5-formyl-(6S)-tetrahydrofolic acid, 5-formyl-(6R)-tetrahydrofolic acid or 5-formyl-(6R,S)-tetrahydrofolic acid, or a pharmaceutically acceptable salt or ester thereof.

15. Method according to claim 11, wherein the at least one folate is selected from 5-methyl-(6S)-tetrahydrofolic acid or 5-methyl-(6R,S)-tetrahydrofolic acid, or a pharmaceutically acceptable salt or ester thereof.

16. Method according to claim 11, wherein the pharmaceutical preparation further comprises one or more additional pharmaceutically acceptable active ingredients and/or adjuvants.

17. Method according to claim 16, wherein the pharmaceutically acceptable active ingredient is aspirin.

18. Method according to claim 16, wherein the pharmaceutically acceptable active ingredient is ascorbic acid.

19. Use of a folate for producing a medicament containing folate as the active agent for improving NO-mediated endothelial-dependent vasomotor responses and reducing vascular superoxide.

20. Use of a folate for producing a medicament containing folate as the active agent for scavenging reactive oxygen species (ROS), increasing vascular BH₄, increasing BH₄/total biopterin ratio, reversing eNOS uncoupling, increasing eNOS dimer:monomer ratio and direct enhancing eNOS activity.

21. Use according to claim 20, wherein the reactive oxygen species (ROS) is peroxynitrite.
